(19) United States
(12) Patent Application Publication
(10) Pub. No.: US 2024/0398871 A1
(43) Pub. Date: Dec. 5, 2024

(54) CELL-FREE LIPOASPIRATE-DERIVED PREPARATION, COMPOSITIONS INCLUDING THE PREPARATION AND USES THEREOF

(71) Applicant: Linio Biotech Ltd, Helsinki (FI)

(72) Inventors: Jertta-Riina Uusmies, Helsinki (FI); Karita Reijonsaari, Helsinki (FI); Veera Hautanen, Helsinki (FI); Enni Sanmark, Helsinki (FI); Timo Ylikomi, Helsinki (FI)

(21) Appl. No.: 18/678,026

(22) Filed: May 30, 2024

Related U.S. Application Data
(60) Provisional application No. 63/505,069, filed on May 31, 2023.

(30) Foreign Application Priority Data
May 31, 2023 (EP) 23176265.9

(51) Int. Cl. A61K 35/35; A61K 8/98; A61K 9/00; A61P 17/02; A61Q 19/08

(52) U.S. Cl. CPC A61K 35/35; A61K 8/981; A61K 9/0019; A61P 17/02; A61Q 19/08

(57) ABSTRACT

A cell-free lipoaspirate-derived preparation, compositions including the same and uses thereof for therapeutic and/or cosmetic treatments of the skin.

### CROSS-REFERENCE TO RELATED APPLICATIONS

This application claims priority to U.S. Provisional Patent Application No. 63/505,069, filed May 31, 2023 and claims priority to European Patent Application No. 23176265.9, filed May 31, 2023, the disclosure of each of these applications is expressly incorporated herein by reference in their entirety.

### TECHNICAL FIELD

The invention belongs to the fields of medicine and cosmetics, in particular to a cell-free lipoaspirate-derived preparation, compositions comprising the same and uses thereof for therapeutic and/or cosmetic treatments of the skin.

### BACKGROUND

The human skin is the outer covering of the body acting as an important protective barrier against sunlight, heat, pathogens, chemicals, injuries and other external factors. Sometimes life renders the skin damaged, aesthetically displeasing and functionally compromised. Over 100 million patients acquire scars from surgeries yearly, and millions suffer from chronic skin conditions and traumatic skin defects. In addition, the skin is affected by aging and environmental factors. This can lead to restricted lifestyles, lower quality of life and mental distress.

Skin regeneration, a natural skin renewal process, is essential to maintaining the health and vitality of the skin, as well as to the healing of traumatic and other skin defects.

Adipose tissue has been proposed as a source for the preparation of various products for different medical and cosmetic purposes. Such products include, for example, nanofat which is obtained by allowing a lipoaspirate to settle such that is separates into three different layers, and by collecting and emulsifying the adipocyte-containing middle layer while discarding the other layers. Such products contain cellular components and thus have limitations in clinical practice. These limitations could potentially be overcome by processing nanofat further by removing its cellular components. For example, Ziyou Yu et al. (Stem Cell Research & Therapy, 2018, 9(1):1-4), Yunfan He et al. (Stem Cell Research & Therapy, 2019, 10(1):1-4), and Yuda Xu et al. (Aesthetic Surgery Journal, 2020, 40(8):904-913) all disclose cell-free products obtained from nanofat.

U.S. Pat. No. 9,631,176 discloses an enzyme-free process for obtaining stem cells from a lipoaspirate.

WO 2011/019822 and Zhaoyang et al. (Journal of Biomedical Materials Research Part A, 220, 109(8):1418-1428) disclose products of processed adipose tissue, which are composed of decellularized adipose tissue extracellular matrix. Such compositions are obtained by chemical or mechanical removal of the cellular compartment of the adipose tissue. The products are thus cell-free and contain little or no lipid remnants, yet having the native architecture of the adipose tissue.

Although some skin regeneration promoting techniques and products have been developed, their effects are still unsatisfactory. Therefore, there remains a well-recognized need for allogenic products that can effectively and safely enhance skin regeneration.

### SUMMARY

In one aspect, the present invention provides a cell-free lipoaspirate-derived preparation comprising at least one of a lipid fraction and an aqueous fraction of a lipoaspirate. In other words, the preparation is free of an adipose tissue fraction of a lipoaspirate, which tissue fraction comprises adipocytes and other cells naturally present in a human adipose tissue.

In another aspect, the present invention provides methods of producing the cell-free preparation of the invention.

In another aspect, the present invention provides a cosmetic and a pharmaceutical composition comprising the cell-free preparation of the invention and a physiologically acceptable carrier, adjuvant and/or excipient.

In a further aspect, the invention provides a method for treating a skin area of a human subject in need thereof, the method comprising: administering the cell-free preparation or the cosmetic preparation of the invention to the skin area topically, subcutaneously, intradermally and/or intrahypodermally.

Further aspects, embodiments and details are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIG. 1 shows photographs of right and left profiles of a male whose temple and cheek areas with acne scars were treated with laser resurfacing with (on the right) or without (on the left) topical administration of the present cell-free lipoaspirate-derived preparation. The photographs illustrate the healing and re-epithelization process 2 days after the treatment.

FIG. 2 shows photographs of a four times operated leg with a three years old scar treated with laser surfacing, taken at different time points (6 h, 2 d, 28 d) after the treatment. The lower half of the scar was treated twice by topical administration of the present cell-free lipoaspirate-derived preparation.

FIG. 3 shows photographs of a skin area with some scars before (on the left) and four weeks after (on the right) a microneedling treatment followed by topical administration of the cell-free lipoaspirate-derived preparation of the invention.

FIG. 4 shows photographs of the eye and mouth areas before (on the left) and four weeks after (on the right) mesotherapy with the cell-free lipoaspirate-derived preparation of the invention.

FIG. 5 shows fibroblast migration as a function of time (hours). Dotted line (A) shows the migration of vehicle control while the solid line (B) shows migration of cell-free lipoaspirate-derived preparation exposure. The significance of difference in each timepoint is shown with asterisks where p-value below 0.01 to 0.001 is shown with one asterisk (*) and p-values below 0.01 to 0.001 are shown with two asterisks (**).

FIGS. 6A and 6B show differential gene expression following 6 days of BJ fibroblast exposure to cell-free lipoaspirate-derived preparation (B) in comparison to vehicle control (A) as fold-change relative to importin 8 (IP08) housekeeping gene. FIG. 6A shows expression of smooth muscle actin (ACTA2), collagen 1 (COL1), collagen 3 (COL3), collagen 8 (COL8) and elastin (ELN), while FIG. 6B shows expression of collagen 4 (COL4A). Asterisks symbolize the significance of differential expression where p-values below 0.05 to 0.01 are shown with one asterisk (*), p-values below 0.01 to 0.001 are shown with two asterisks (**), and p-values below 0.001 are shown with three asterisks (***).

FIG. 7 shows triglyceride formation in human adipose stem cells with vehicle control (A) with two different concentrations exposure to cell-free lipoaspirate-derived preparation; 0.8 mg/ml (B) and 0.4 mg/ml (C), with significance of change calculated against vehicle control. Significances are shown as asterisks where p-values below 0.05 to 0.01 are shown with one asterisk (*) and p-values below 0.01 to 0.001 are shown with two asterisks (**).

FIG. 8 shows differential gene expression following 6 days of human adipose stem cell exposure to cell-free lipoaspirate-derived preparation (B) in comparison to vehicle control (A). FIG. 8 shows expression of smooth muscle actin (ACTA2) and collagen 1 (COL1). Significances are shown as asterisks where p-values below 0.05 to 0.01 are shown with one asterisk (*).

FIG. 9 shows increased keratinocyte proliferation following 6 days of exposure to cell-free lipoaspirate-derived preparation (B) normalized against control (A), where the control receives value 1. The significance of difference is shown with asterisks where p-value below 0.01 to 0.001 is shown with one asterisk (*).

FIG. 10 shows keratinocyte migration as a function of time (days). Dotted line (A) shows migration of vehicle control while the solid line (B) shows migration of cell-free lipoaspirate-derived preparation exposure. The significance of difference in each timepoint is shown with asterisks where p-value below 0.01 to 0.001 is shown with one asterisk (*) and p-values below 0.01 to 0.001 are shown with two asterisks (**).

FIGS. 11A, 11B and 11C show differential gene expression following 15 days of keratinocyte exposure to cell-free lipoaspirate-derived preparation (B) in comparison to vehicle control (A).

FIG. 11A shows expression of epidermal growth factor (EGF), FIG. 11B expression of matrix metalloproteinase 1 (MMP1) and FIG. 11C expression of TIMP metallopeptidase inhibitor 1, a tissue inhibitor of metalloproteinases 1 (TIMP-1). Significances are shown as asterisks where p-values below 0.05 to 0.01 are shown with one asterisk (*).

FIG. 12 shows selected enrichment terms from Gene Ontology (GO) sub-ontologies; Biological Processes (BP), cellular compartments (CC) and molecular functions (MF). X-axis shows 10-logarithmic value of p-value calculated in the enrichment analysis and the dot size shows number of genes in each term. The GO terms in y-axis are: 1=angiogenesis, 2=regulation of peptidase activity, 3=tissue migration, 4=immune system process, 5=fatty acid metabolic process, 6=response to oxidative stress, 7=blood coagulation, 8=regulation of cellular component organization, 9=wound healing, 10=cellular detoxification, 11=supramolecular fiber organization, 12=proteolysis, 13=immunoglobulin complex, 14=high-density lipoprotein particle, 15=extracellular matrix, 16=collagen-containing extracellular matrix, 17=extracellular exosome, 18=lipid binding, 19=glycosaminoglycan binding, 20=extracellular matrix structural constituent, 21=antioxidant activity, 22=peptidase regulator activity, 23=oxidoreductase activity, 24=cytoskeletal protein binding, 25=cell adhesion molecule binding.

### DETAILED DESCRIPTION

The following detailed description illustrates aspects and embodiments of the present invention and ways in which they can be implemented. Although some modes of carrying out the present invention have been disclosed, it is to be understood that other embodiments for carrying out or practicing the present invention are also possible. In other words, the scope of the present invention will be limited only by the appended claims.

It is also to be understood that the terminology used herein has the same meaning as commonly understood by one of ordinary skill in the art to which this invention belongs, unless otherwise specified.

As used herein, a singular noun, unless otherwise specified, carries also the meaning of the corresponding plural noun. In other words, the singular expressions “a”, “an” and “the” carries not only the meaning of “one” but also “one or more”, unless otherwise specified.

The term “and/or” in a phase such as “X and/or Y” shall be understood to mean either “X and Y” or “X or Y” and shall be taken to provide explicit support for both meanings or for either meaning.

The terms “comprising”, “including”, “containing” and “having” can be used interchangeably, and are intended to be construed in a non-exclusive manner, namely allowing for items, components or elements not explicitly described also to be present.

In one aspect, the present invention provides a cell-free preparation comprising or consisting of a lipid fraction and/or an aqueous fraction of a lipoaspirate. Accordingly, the terms “cell-free preparation” and “cell-free lipoaspirate-derived preparation” as used herein are interchangeable, and sometimes called “preparation” for short in this description.

As used herein, the term “cell-free” refers to a preparation that is devoid or essentially devoid of cells. Techniques for determining whether a given preparation is cell-free or not are readily available in the art.

As used herein, the term “lipoaspirate” refers to material obtainable by liposuction. It may originate from a single human subject or it may be a mixture of lipoaspirates originating from more than one human subject. Both options are included in the term “a lipoaspirate”.

Liposuction is a surgical removal of subcutaneous adipose tissue (i.e., hypodermal adipose tissue) by means of aspiration cannulas introduced through small skin incisions, assisted by suction. Various liposuction techniques are readily available and have evolved as technology has improved. Technological advancements added to the traditional dry liposuction technique include ultrasonic liposuction, water-assisted liposuction, and radiofrequency-assisted liposuction to name some non-limiting examples. New state-of-the-art liposuction technique is water-assisted liposuction, a technique that utilizes a pressurized stream of saline/aqueous buffered solution in an amount that is about two to three times larger than the volume of subcutaneous fat to be removed, thereby causing the fatty subcutaneous tissue layer to swell and become firm prior to aspiration and to ease the removal of subcutaneous fat/tissue from different areas in the body. Commonly used saline/aqueous buffered solution in water-assisted liposuction is called “tumescent solution”. Accordingly, the liposuction technique can be called tumescent liposuction. Traditional tumescent solution contains local anesthetics such as lidocaine and adrenaline or epinephrine. Notably, the present invention is not limited to any particular liposuction technique, as the choice of the technique often depends on patient characteristics and surgeon preference.

As used herein, the term “liposuction solution” refers to any physiologically acceptable aqueous solution suitable for use as an infiltrate in any liposuction technique, including not only the conventional tumescent liposuction but also other water-assisted liposuction techniques. Non-limiting examples of suitable liposuction solutions include 0.9% sodium chloride (saline), Ringer Acetat, Ringer fundin, Ringer lactate and Normofundin. Those skilled in the art can easily select an appropriate aqueous buffered solution for each case. In some embodiments, the liposuction solution may contain any approved local anesthetic, such as lidocaine or prilocaine, typically 0.5% or 1% lidocaine (up to 55 mg/kg). In some other embodiments, the liposuction solution may contain adrenaline/epinephrine, typically 1% adrenaline (up to 7 mg/kg), for temporary vasoconstriction to prevent bleeding during liposuction. In some embodiments, the liposuction solution may contain both lidocaine (and/or any other appropriate local anesthetic) and adrenaline, preferably in amounts mentioned above. Such a liposuction solution is traditionally called tumescent solution, although any buffered aqueous solution used in liposuctions is sometimes called as “tumescent solution”, regardless of whether lidocaine or adrenaline/epinephrine is present or not. Therefore, the terms “liposuction solution” and “tumescent solution” as used herein are interchangeable, unless otherwise indicated. It follows that in some further embodiments, a liposuction liquid containing neither lidocaine (or any other local anesthetic) nor adrenaline/epinephrine may still be called as “tumescent solution”.

The term “spent liposuction solution” refers to a liposuction solution that has been used in liposuction, i.e. that has been in contact with human subcutaneous fat in vivo and/or after aspiration ex vivo. The spent liposuction solution comprises various at least partly water-soluble substances originating from the subcutaneous fat tissue.

When a lipoaspirate obtained by a liposuction technique that involves use of a liposuction solution is centrifuged or allowed to stand for a while, the lipoaspirate separates into different layers, namely into a lipid layer on the top, an adipose tissue layer comprising adipocytes and other cells of the adipose tissue in the middle, and an aqueous layer at the bottom. The aqueous layer contains the spent liposuction solution used for the liposuction. Any cell or tissue debris contained in the lipoaspirate may separate into a fourth layer below the aqueous layer. In this context, the term “layer” can be used interchangeably with the term “fraction”.

As used herein, the term “fraction” is not limited to the whole fraction (i.e., layer) in question, but includes also any portions thereof. In other words, the cell-free preparation of the invention need not contain the whole lipid fraction and/or the whole aqueous fraction of a lipoaspirate. To put it differently, cell-free preparations containing only a portion of the lipid fraction and/or a portion of the fraction of a lipoaspirate are encompassed by the claimed cell-free lipoaspirate-derived preparation comprising at least one of a lipid fraction and an aqueous fraction of a lipoaspirate.

The cell-free preparation of the invention is obtainable by a method in which a lipoaspirate is processed in a manner that results in a preparation that contains a lipid fraction and/or an aqueous fraction of the lipoaspirate, and is devoid of cells naturally present in an adipose tissue. This may be achieved in different ways. In some embodiments, the method comprises the steps of removing the adipose tissue fraction of a lipoaspirate, collecting the remaining lipid fraction and/or aqueous fraction, and sterile filtering the collected fraction(s). In some other embodiments, the adipose tissue fraction is not actively removed but only the desired fractions, i.e., either the lipid fraction, the aqueous fraction, both are collected and subsequently sterile-filtered. Since no washing steps are carried out prior to collecting the aqueous fraction, it contains the spent liposuction solution. It follows, that the collected fraction may or may not contain a local anesthetic, such as lidocaine, and/or adrenaline. It also follows that the lipid fraction is not removed by any washing steps. In some embodiments, the lipoaspirate is shaken gently and/or allowed to settle such that it separates into different layers prior to removing the adipose tissue fraction and/or collecting the lipid/or the aqueous fraction. The adipose tissue fraction can be removed by any suitable technique available in the art. Also collecting of the lipid fraction and/or the aqueous fraction can be carried out by any suitable technique available in the art. Sterile filtering is usually carried out by using a 0.22 μm filter, i.e. a membrane filter having a pore size of 0.22 μm. Alternatively, sterile filtering may be carried out using a 0.2 μm filter, i.e. a membrane filter having a pore size of 0.2 μm.

In some embodiments, liposuction solution used in the liposuction but not aspirated together with the adipose tissue (i.e., is collected to a separate container during or right after the liposuction) may be provided as the present cell-free preparation or used as an aqueous component thereof, for example by way of being added to the collected aqueous fraction of a lipoaspirate to increase its volume. In some further embodiments, any physiologically acceptable aqueous buffered solution/saline may be added to the lipoaspirate to increase the volume of the aqueous fraction, if desired.

In some embodiments, the spent liposuction solution, either as a collected aqueous fraction of a lipoaspirate or as a spent liposuction solution not forming part of a lipoaspirate may be diluted or concentrated as desired by adjusting its volume using physiologically acceptable diluents and/or techniques readily available in the art. In some embodiments, the spent liposuction solution may be freeze-dried and reconstituted on demand.

In accordance with the above, the term “aqueous fraction of a lipoaspirate” refers to the bottom fraction of a lipoaspirate or the bottom fraction of a spent liposuction solution, which bottom fractions form when said lipoaspirate or said spent liposuction solution that has been allowed to settle, i.e. separate into layers described above.

In some embodiments, the preparation of the invention consists of a lipid fraction of a lipoaspirate. In some embodiments, the preparation comprises a lipid fraction of a lipoaspirate as the only lipoaspirate-derived material. In some further embodiments, the preparation provided as a filtered, preferably a sterile-filtered lipid fraction of the lipoaspirate. In some still further embodiments, the lipid fraction has been sterile-filtered through a 0.2 μm filter or a 0.22 μm filter. In some even further embodiments, the lipid fraction may be provided as a freeze-dried preparation that is to be reconstituted on demand.

In some embodiments, the preparation of the invention consists of an aqueous fraction of a lipoaspirate and/or of a spent liposuction solution not being part of a lipoaspirate. In some embodiments, the preparation of the invention comprises an aqueous fraction of a lipoaspirate and/or a spent liposuction solution not collected as part of the lipoaspirate, as the only liposuction-derived material in the preparation. In some further embodiments, the preparation is a filtered, preferably a sterile-filtered lipid fraction of a lipoaspirate and/or of a spent liposuction solution not being part of a lipoaspirate. In some still further embodiments, the preparation has been sterile-filtered through a 0.2 or a 0.22 μm filter. In some even further embodiments, the preparation is provided as a freeze-dried preparation that is to be reconstituted on demand.

In some embodiments, the preparation of the invention consists of a lipid fraction and an aqueous fraction of a lipoaspirate with or without being supplemented with a spent liposuction solution not being part of the lipoaspirate. In some embodiments, the preparation of the invention comprises a lipid fraction and an aqueous fraction of a lipoaspirate with or without being supplemented with a spent liposuction solution not collected as part of the lipoaspirate, as the only liposuction-derived material in the preparation. The lipid fraction may be the whole lipid fraction of the lipoaspirate, or a portion thereof. In some further embodiments, the preparation consists of filtered, preferably sterile-filtered lipid and aqueous fractions of a lipoaspirate. In some still further embodiments, the preparation has been sterile-filtered through a 0.2 μm or a 0.22 μm filter. In some even further embodiments, the preparation may be provided as a freeze-dried preparation that is to be reconstituted on demand.

In some embodiments, the preparation of the invention comprises or consists of a spent liposuction solution without the adipose tissue fraction, i.e. without the middle fraction that forms when a lipoaspirate or a spent liposuction solution is allowed to settle. In some embodiments, the preparation of the invention comprises a spent liposuction solution without the adipose tissue fraction, i.e. without the middle fraction that forms when a lipoaspirate or a spent liposuction solution is allowed to settle, as the only liposuction-derived material in the preparation. In some further embodiments, the preparation is provided as a filtered, preferably sterile-filtered preparation. In some still further embodiments, the preparation has been sterile-filtered through a 0.2 μm or a 0.22 μm filter. In some even further embodiments, the preparation may be provided as a freeze-dried preparation that is to be reconstituted on demand.

In any of the above-described embodiments, the preparation may have been supplemented with a spent liposuction solution which is not part of the actual lipoaspirate.

In accordance with what is described above, the preparation of the invention is obtainable by a method comprising the steps of:
providing a lipoaspirate obtained from at least one human subject;
optionally, shaking the human lipoaspirate;
removing an adipose tissue fraction of the lipoaspirate, said fraction comprising cells; and
filtering the remaining fractions of the lipoaspirate.

In some further embodiments of the above method, the lipoaspirate is allowed to settle prior to removal of the adipose tissue fraction, such that the lipoaspirate separates into different layers, namely into a lipid layer on the top, an adipose tissue layer comprising adipocytes and other cells of the adipose tissue in the middle, and an aqueous layer at the bottom.

In some other embodiments, the preparation of the invention is obtainable by a method comprising the steps of:
providing a lipoaspirate obtained from at least one human subject,
optionally, shaking the human lipoaspirate;
collecting at least one of a lipid fraction and an aqueous fraction, while discarding an adipose tissue fraction comprising cells; and
filtering the collected fraction(s).

In some further embodiments of the above method, the lipoaspirate is allowed to settle prior to collecting at least one of a lipid fraction and an aqueous fraction, such that the lipoaspirate separates into different layers, namely into a lipid layer on the top, an adipose tissue layer comprising adipocytes and other cells of the adipose tissue in the middle, and an aqueous layer at the bottom. The lipid and/or the aqueous fraction may be collected at least partly.

In the methods, filtering of the remaining or collected fractions limits the particle size of various constituents in the resulting cell-free preparation, depending on the pore size of the filter used. If a 0.2 μm or a 0.22 μm filter is used, the preparation of the invention does not contain large intact ECM proteins having at least one dimension that is larger than 0.2 μm or 0.22 μm, respectively. Filtering the remaining or collected fractions with such a filter also makes the preparation sterile. Therefore, a preferred filter is a 0.2 μm filter or a 0.22 μm filter.

Notably, obtaining of the lipoaspirate from the at least one human subject is not part of the above-described methods. In other words, the methods are to be applied on a lipoaspirate obtained from said at least one human subject earlier.

As aspect of the present disclosure relates to the above-described methods as such.

Being cell-free means that the present preparation is fundamentally different from any adipose tissue-derived stromal vascular fraction (SVF) which contains heterogeneous cell populations such as mesenchymal progenitor/stem cells, preadipocytes, endothelial cells, pericytes, T cells, and M2 macrophages. The SVF is obtainable from the adipose tissue fraction of a lipoaspirate. Accordingly, the present preparation is also fundamentally different from nanofat, which is an emulsified and filtered adipose tissue fraction of a lipoaspirate, as well as from any cell-free extracts obtained from nanofat by removal of the cells. In other words, nanofat is obtained by discarding the lipid and the aqueous fractions of a lipoaspirate, and by emulsifying the remaining adipose tissue fraction, i.e. the cell fraction. It is to be understood that even if the emulsified cell fraction is processed further by removal of the cellular components, which process may involve use of an aqueous solution, the resulting cell-free extract does not qualify as the present aqueous fraction of a lipoaspirate, even if the nanofat-derived cell-free extract was aqueous.

Moreover, the present cell-free preparation is fundamentally different from any decellularized fat-derived compositions available in the art. Such decellularized compositions are natural scaffolds derived from adipose tissue, in which the cellular and nuclear contents are eliminated, but the three-dimensional structure and composition of the extracellular matrix (ECM) are preserved.

The preparation of the invention has several advantages by way of being cell-free, including for example, low immunogenicity and hence reduced risk of adverse effects when administered to a human body. It follows that the preparation of the invention may be provided as an allogenic product, i.e., as a product suitable for use by subjects different from the donor of the lipoaspirate. There are also many practical benefits when factors such as cell viability during storage or after administration need not be considered.

Earlier studies have indicated that an adipose tissue-derived cell-free extract can be used for soft tissue engineering and repair owing to its ability to induce both angiogenesis, i.e., formation of new blood vessels, and adipogenesis, i.e., formation of new adipose tissue.

It has now been unexpectedly realized that the cell-free lipoaspirate-derived preparation of the present invention is capable of promoting skin regeneration, a process that does not involve adipogenesis and that is affected not only by angiogenesis but is largely dependent also on other cellular processes such renewal of epidermal and dermal cells, especially activation of keratinocytes and fibroblasts, respectively.

Accordingly, in some embodiments, the present cell-free preparation comprising or consisting of a lipid fraction and/or an aqueous fraction of a lipoaspirate may be denoted as a cell-free allogenic skin regeneration substitute.

Skin is the largest organ of the human body, consisting of three layers. The outermost layer of skin is epidermis, a thin protective layer consisting primarily of keratinocytes. The middle layer of skin is dermis, which makes up 90% of skin's thickness. The dermis is rich in collagen, a protein that makes skin cells strong and resilient, and in elastin, a protein that keeps the skin flexible and helps stretched skin regain its shape. The basement membrane zone between the epidermis and the dermis connects, and functionally separates, the epidermis and the dermis, and is indispensable for normal skin functions. The bottom layer of skin is hypodermis (i.e., subcutis), a fatty layer that cushions underlying muscles and bones. Hypodermal fat is arranged in the form of lobules separated from each other by fibrous septae that consist of blood vessels, nerves, lymphatics and connective tissue. Each lobule contains adipocytes (i.e., fat cells), which consist mostly of triglycerides.

As used herein, the term “hypodermal” refers to a matter derivable from human hypodermal tissue, i.e. subcutaneous tissue. The term may be used interchangeably with the terms “hypodermis-derived” or “subcutis-derived”.

Since the preparation of the invention is derived from a lipoaspirate, it may also be denoted, for example, as a cell-free hypodermal preparation.

As used herein, the term “skin regeneration” refers to a natural process that occurs as the skin cells turn over. In other words, dead skin cells on the top layer of the epidermis fall away, revealing fresh, newly created cells beneath. Scar tissue forms when skin heals but it doesn't regenerate. As aging slows down the skin regeneration and changes the basement membrane zone, the skin becomes less elastic, thinner and more wrinkled or textured.

Without being limited to any theory, the preparation of the invention may promote skin regeneration, thereby improving management of scars and other skin defects, as well as diminishing visible signs of skin aging, by a number of different mechanisms of action. For example, the preparation not only brings essential structural components (i.e., building blocks) and nutrients to the skin but also induces the local cells to produce more essential factors. More specifically, as demonstrated in the examples, the preparation activates fibroblasts, cells that produce collagen, hyaluronic acid, and elastin, as well as induces local cell migration and differentiation. It is also demonstrated in the examples that the preparation activates keratinocytes and increases their turnover, thereby improving the barrier function of the skin. Moreover, the preparation is envisaged to improve functioning of the dermal-epidermal junction and repair thereof.

Aging skin becomes drier and thinner, loses its firmness and appears more wrinkled. The moisture content decreases in the outermost layer of the skin, which is partly due to the reduction of lipids, i.e. fatty substances, and partly due to decrease in hyaluronic acid. Both of these changes affect the skin such that it is no longer able to bind and retain water in the same way as a younger skin does. The cell-free preparation of the invention contains a wide number of different lipids, fatty acids, associated proteins, and proteins related to lipid metabolism, which are considered to help the skin retain moisture thereby reducing its dryness, thereby not only rejuvenating aged skin but also providing means to manage clinical and other skin conditions and defects that benefit from reduced dryness of the skin.

To be more specific, based on untargeted compositional analysis of the preparation, it essentially contains lipids, associated proteins and proteins related to lipid metabolism such as but not limited to ceramids, apolipoproteins, perilipins, non-saturated and saturated fatty acids, glycerophospholipids, lysophosphatidic acids, lysophospholipids, monoglycerides, diglycerides, triglycerides and prostaglandins. Some of these molecules may originate from the lipid fraction of a lipoaspirate, while some other molecules may originate from the aqueous fraction of a lipoaspirate. Accordingly, some of the components in the preparation may be fat-soluble while some other components may be water-soluble or partly water-soluble. Ceramics are particularly interesting components of the preparation according to the invention because they are lipids found in normal skin cells.

In accordance with the other proposed mechanisms of action, characterization of the preparation of the invention also revealed that it essentially contains also structural and non-structural extracellular matrix proteins, peptides, chains, subchains and/or subunits thereof, as well as proteins related to the synthesis of extracellular matrix. These essentially include but are not limited to Collagens I, III, IV, VI, XV, XVIII, fibronectin, vitronectin, elastin, hyaluronan, decorin, tenascin, laminin, lumican and prolargin. Interestingly, lumican is a leucine-rich repeat proteoglycan that induces fibrillogenesis of collagen, whereas prolargin is a protein that anchors the basement membrane to the underlying tissue.

Interestingly, the preparation of the invention contains basement membrane proteins peptides, chains, subchains and/or subunits thereof, as well as specialized dermo-epidermal junction components, and factors effecting epidermis, including for example, without limitation, keratin, nidogen, versican, intergrins, periplakin, and plectin.

It is to be understood that although the present preparation may contain components of the ECM and the basement membrane (BM), it contains neither intact ECM nor intact BM, and is thus fundamentally different from any decellularized compositions. In this context, intact ECM and/or BM refers to compositions, in which the three-dimensional structure and composition of the ECM and/or BM are preserved, respectively. The preparation does not contain distinct large components of the ECM and/or BM either. As an example, the preparation does not contain intact collagen fibrils, but may contain different collagen molecules, microfibrils or subchains. Collagen exists in different forms, including at least collagens I, III, IV, VIII, and in different molecular sizes. The method for producing the preparation of the invention does not involve extraction of collagens or other large ECM/BM components, and does not even enable the presence of such intact components in the resulting preparation.

Moreover, in those embodiments, which involve filtering through a 0.2 μm or a 0.22 μm filter, no components larger in size than said pore size can be present. It is to be noted that intact collagen I fibrils, for example, can be significantly larger in size than the pore size of a 0.2 μm filter or a 0.22 μm filter. Thus, although the filtered preparation may contain various peptides, side chains and microfibrils of collagens and other ECM/BM components, their molecular size is limited to 200 nm or 220 nm.

Notably, the preparation of the invention also contains essential and non-essential amino acids, antioxidants, oxidoreductases, vitamin derivatives and metabolites, and wide number of glycoproteins and proteoglycans as well as factors related to cell proliferation. Also these findings are in accordance with the proposed mechanisms of action, although the present invention is not limited to any theory or mechanism of action. Tretinoin is a particularly interesting component of the present preparation because it is an antioxidant that is famous for its anti-aging benefits and therefore often added into various skin care products. Tretinoin may also be classified into vitamins, more specifically vitamin derivatives or metabolites.

Surprisingly, neither VEGF, IGF-1 nor FGF-2 was among over 1004 proteins, over 6000 related peptides and over 500 metabolites identified as components of the present preparation on the basis of untargeted analysis by liquid chromatography-mass spectrometry (LC-MS).

The cell-free preparation of the invention may be applied for various therapeutic and/or cosmetic treatments of the skin. It may be used as such or as formulated into a pharmaceutical or a cosmetic composition.

As used herein, the term “pharmaceutical composition” refers broadly to a composition comprising the present cell-free lipoaspirate-derived preparation as a therapeutically active ingredient and one or more pharmaceutically acceptable components such as carriers, adjuvants and/or excipients. As used herein, the term “pharmaceutically acceptable” refers to a material that is suitable for administration to a human subject without undue adverse side effects such as toxicity, significant irritation and/or allergic responses. In other words, the benefit/risk ratio must be reasonable. In essence, the term “pharmaceutically acceptable” is interchangeable with the term “physiologically acceptable”. Moreover, the pharmaceutically acceptable component should be such that it does not diminish the therapeutic activity of active ingredient, i.e., the present lipoaspirate-derived preparation.

As used herein, the term “cosmetic composition” refers broadly to a composition comprising the present cell-free lipoaspirate-derived preparation as an active ingredient and one or more cosmetically acceptable components such as carriers, adjuvants and/or excipients. As used herein, the term “cosmetically acceptable” refers to a material that is suitable for administration to a human subject without undue adverse side effects such as toxicity, significant irritation and/or allergic responses. In other words, the benefit/risk ratio must be reasonable. As readily understood by those skilled in the art, in order to be “cosmetically acceptable” the composition must also be “physiologically acceptable” as well as “dermatologically acceptable”. Therefore, the terms can be used interchangeably.

Those skilled in the art to which the present invention belongs, can readily select appropriate pharmaceutically and/or cosmetically acceptable components available in the art, depending on the intended route of administration and formulation of the composition. The formulation may be carried out as desired using means and methods readily available in the art, for example by means of conventional mixing, dissolving, granulating, levigating, emulsifying, encapsulating, entrapping, lyophilizing or the like.

Excipients are preferably inert substances added to pharmaceutical and cosmetic compositions. Typical examples of different types of excipients, without limitation, include stabilizers, preservatives, pH modifiers, fillers, thickeners, viscosity modifiers, lubricants, solubilizers, surfactants, and the like.

Pharmaceutical and cosmetic compositions for topical administration include, but are not limited to, ointments, lotions, creams, gels, hydrogels, oil-in-water emulsions, water-in-oil emulsions, drops, sprays, liquids, solutions, powders and slow release or sustained release formulations, face masks, skin patches, mousses and foams.

Pharmaceutical and cosmetic compositions for parenteral administration are typically sterile aqueous or non-aqueous solutions, suspensions or emulsions to be applied topically or by injection, for example subcutaneously, intrahypodermally or intradermally. In some embodiments, the composition may be administered into different layers of the skin during the same treatment session and/or process. For example, the composition may be administered both by a subcutaneous, intrahypodermal and/or intradermal injection and by topical administration.

The composition, be it a pharmaceutical or a cosmetic composition, may also be provided in a concentrated form or in a form of a powder (i.e. lyophilized) to be re-constituted on demand, regardless of whether the composition is intended for topical or parenteral administration.

The present invention provides the cell-free lipoaspirate-derived preparation disclosed herein for use in aesthetic dermatology. This aspect of the invention may be expressed, for example, as a use of said cell-free preparation or a cosmetic composition comprising the same for cosmetic treatment of the skin or as a non-therapeutic method for cosmetically treating the skin, more specifically a selected area of the human skin. The method comprises a step of applying said preparation or said composition to the skin to be treated, preferably in a cosmetically efficient amount. In some embodiments, the skin to be treated is compromised skin.

As used herein, the expressions “cosmetic treatment” and “non-therapeutic treatment” may be used interchangeably, both referring to the administration of the present cell-free preparation or a cosmetic composition comprising the same to a subject in need thereof for a purpose which may include promoting skin regeneration and/or skin rejuvenation. Improved skin regeneration and/or rejuvenation may manifest itself, without limitation, as reduced dark circles around the eyes, reduced dryness and roughness of the skin, reduced wrinkles and fine lines, decreased redness of the skin, reduced number, size and/or darkness of age spots (liver spots) and other hyperpigmentation defects, reduced number, size and/or whiteness of vitiligo patches, decreased cellulite, burns, improved stretch marks and scars, such as scars associated with acne or other clinical skin conditions, or scars caused by injuries or operations.

As used herein, the term “cosmetically efficient amount” refers to an amount by which signs of cosmetic defects, such dark areas around the eyes, dryness and roughness of the skin, wrinkles and fine lines, redness of the skin, age spots (liver spots) and other hyperpigmentation defects, vitiligo patches, cellulite, stretch marks and scars, are at least reduced or ameliorated.

As used herein, the term “compromised skin” refers to the skin whose outer layer, i.e., the epidermis, has been damaged for any reason. In relation to aesthetic dermatology, the skin to be treated may be comprised owing a cosmetic treatment such as microneedling, laser treatment or exfoliation, or owing to aging.

In a further aspect, the present invention provides the cell-free preparation disclosed herein for use in clinical dermatology. This aspect of the invention may be expressed, for example, as a cell-free preparation or a pharmaceutical composition comprising the same for use in treating a clinical skin disorder or condition, or as a therapeutic method for treating a clinical skin disorder or condition, typically exhibiting compromised skin. The method comprises a step of applying the preparation or the composition to the skin to be treated, preferably in a therapeutically efficient amount.

As used herein, the expression “therapeutic treatment” in its different linguistic forms refers to the administration of the present cell-free preparation or a pharmaceutical composition comprising the same to a subject in need thereof for a purpose which may include ameliorating, lessening, inhibiting, or curing a clinical skin disorder or condition.

As used herein, the term “clinical skin condition” refers to a clinical dermatologic disorder, condition or defect that typically manifests itself as compromised skin, and often exhibits an undesirable, unsightly skin appearance. Such skin conditions often involve inflammation, such as that caused by a bacterial, fungal or viral infection. Clinical skin conditions that are envisaged to benefit from improved skin regeneration by the present cell-free preparation or a pharmaceutical or cosmetic composition comprising the same include, but are not limited to Acne, Actinic Keratosis, Atopic Dermatitis, venous stasis dermatitis, eczema, Basal Cell Carcinoma, Contact Dermatitis, Keloids, Lichen Planus, Melasma, Vitiligo, Psoriasis, Rosacea, Seborrheic Dermatitis, acute and chronic wounds such as diabetic wounds, burns, Erythema Multiforme, Epidermolysis bullosa, Lupus and keratosis pilaris.

Clinical skin conditions may also manifest excess fibrosis, the pathological accumulation of excess fibrous connective tissue. It is a common response to chronic injury. Without being limited to any theory, the present cell-free preparation is capable of reversing scarring and fibrosis at least by being able to induce normal cellular functions in scars and in other fibrotic areas, for example through activation of fibroblasts.

The ability of the cell-free preparation of the invention to reverse scarring and fibrosis is not limited to the skin as the application area. It is therefore envisaged that the preparation is suitable for treating also other fibrotic conditions than fibrotic skin conditions and hypertrophic scars such as chronic and acute wounds and burns as well as other skin injuries, keloids, and keratosis pilaris. Non-limiting examples of such other fibrotic conditions include Peyronie's disease, Urethral stricture, and Abdominal adhesions. Accordingly, provided herein is also use of the present preparation for the treatment of fibrotic conditions, including not only fibrotic skin conditions but also other fibrotic conditions, as well as methods for said treatment in a subject in need thereof, the method comprising administration of the present preparation in accordance with what is described elsewhere in this description.

As used herein, the term “therapeutically efficient amount” refers to an amount by which harmful effects of the skin condition are, at a minimum, ameliorated.

Those skilled in the art will readily understand that cosmetic and therapeutic treatments may often overlap. Moreover, some skin conditions may be considered as both clinical dermatological conditions or defects and aesthetic skin conditions and defects. Both types of skin conditions and defects, regardless of how they are classified, benefit from the treatment with the preparation of the invention.

In some embodiments of the above-mentioned therapeutic and/or cosmetic uses and methods, the present preparation or a composition comprising the same is to be applied topically onto the skin to be treated, such as onto compromised skin. In some embodiments, said methods may comprise the steps of i) preparing compromised skin, for example by a treatment that involves microneedling, exfoliation or laser resurfacing, and ii) applying the preparation or the composition on the compromised skin so prepared. In some other embodiments, said methods may comprise a step of applying the preparation or the composition on pre-compromised skin, i.e. on skin that has been prepared earlier, for example by any of the treatments mentioned above, said compromising treatments thus not forming part of the therapeutic and/or cosmetic treatment of some embodiments of the invention.

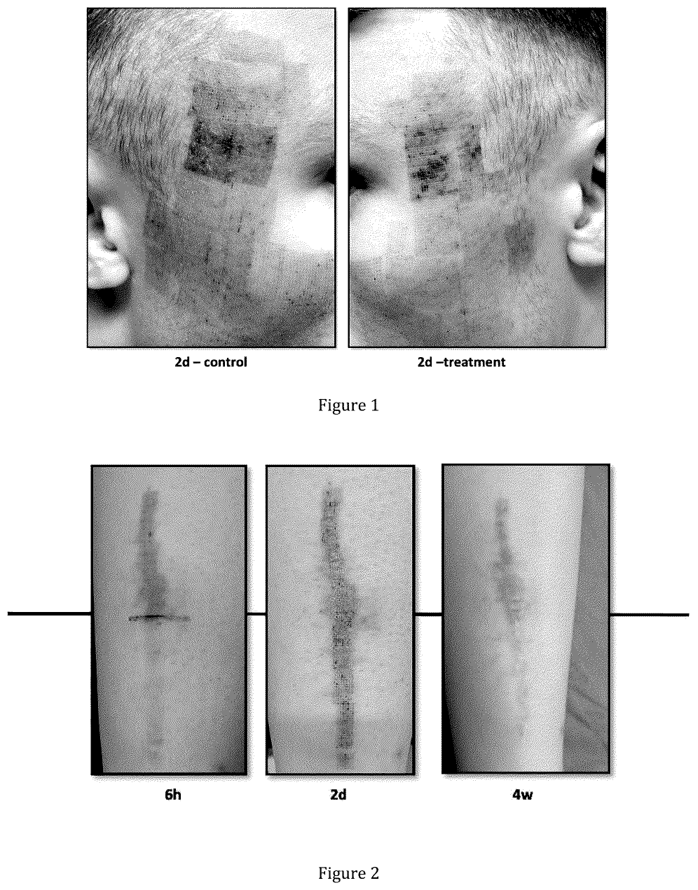

In accordance with what is stated above, when the preparation of the invention is applied to skin areas associated with acne or other scars, preferably compromised e.g. by laser resurfacing or preferably by any intradermal needling techniques known to those skilled in the art, the healing process becomes faster, the scars fade and a healthy skin appearance is observed. Indeed, faster healing process and improved scarring reversal in response to administration of the preparation of the invention onto laser resurfaced facial areas associated with acne scars is demonstrated in FIG. 1, whereas activation of acne scar repair in response to administration of the preparation of the invention by intradermal injections is demonstrated in Example 7. Moreover, fading of scars and improved skin appearance is demonstrated in FIG. 2. To conclude, these results show that preparation of the invention has the potential to promote skin regeneration, especially in scarred skin. Further evidence regarding the potential of the present preparation to reverse scaring and skin fibrosis is provided in Examples 8, 9 and 10. These examples demonstrate that the present preparation is capable of activating fibroblasts, inducing mesenchymal stem cells and activating keratinocytes, respectively.

Furthermore, when the preparation of the invention is applied to skin areas, preferably compromised e.g. by microneedling, reduction of wrinkles, pores, UV spots, brown spots and red areas is observed. These results show that preparation of the invention has the potential to promote skin regeneration and/or skin rejuvenation.

In some further embodiments of the above-mentioned therapeutic and/or cosmetic uses and methods, the present preparation or a composition comprising the same is to be applied intradermally into the skin to be treated, such as acne scars or aged skin or photodamaged skin, for example as mesotherapy. Also in such embodiments, improvement in skin quality, health and appearance is observed as judged, without limitation, by reduction of wrinkles, pores, UV spots, brown spots and red areas. Also these results demonstrate that preparation of the invention has the potential to promote skin regeneration and/or skin rejuvenation.

In one aspect, the present invention provides use of the cell-free preparation disclosed herein for the manufacture of a cosmetic composition for the non-therapeutic treatment of skin, such as compromised or non-compromised skin including aged skin and scarred skin, as well as for the manufacture of pharmaceutical composition for the therapeutic treatment of a clinical conditions or defects, such as clinical skin conditions.

In a further aspect, the present invention provides use of the cell-free preparation or the cosmetic or the pharmaceutical preparation disclosed herein for promoting one or more of the following effects; activation of keratinocytes, activation of fibroblasts, induction of mesenchymal stem cells, induction of endogenous production of collagen I, collagen III, collagen IV, collagen VIII, elastin, EGF and/or MMP-1. A corresponding method for promoting said effects is also provided, the method comprising administration of the cell-free preparation, the cosmetic composition or the pharmaceutical composition an area of a human body in which promotion of one or more of said effects is desired.

### Example 1. Preparation and Characterization a Cell-Free Hypodermal Preparation

Human lipoaspirate samples were obtained with a signed informed consent from three healthy volunteers undergoing standard water-assisted liposuction.

The lipoaspirates were processed without rinsing by first subjecting them to gentle shaking, and then by allowing the shaken lipoaspirates to separate into a lipid fraction on the top, an adipose tissue fraction containing adipocytes and other cells in the middle, and an aqueous fraction containing the used liposuction solution at the bottom. The middle fraction was removed, whereas the lipid fraction and the aqueous fraction were collected and combined, followed by sterile filtering using a 0.22 μm filter. The cell-free preparations so obtained were stored at −20° C. in aliquots.

Three independent patches from three different donors so obtained were subjected to untargeted metabolomics analysis, a comprehensive analysis of all the measurable analytes in a sample including chemical unknowns, by using UPLC-MS platform.

Sample preparation was carried out as follows: samples of the three batches were thawed on ice, followed by transferring 100 μL of each sample into a new tube and addition of 300 μL methanol. Next, the samples were vortexed for 30 s, treated by sonication for 30 min at 4° C., and kept at −20° C. for 1 hour. Next, the samples were vortexed for 30 s, and kept at −20° C. for 0.5 h. Thereafter, the samples were centrifuged at 12000 rpm for 15 min at 4° C. Finally, 200 μL of supernatant and L of DL-o-Chlorophenylalanine (0.5 mg/mL) was transferred to avial for LC-MS analysis.

LC-MS Analysis was carried out as follows: separation was performed by Waters Acquity UPLC combined with Q Exactive MS (Thermo) and screened with ESI-MS. The LC system was comprised of ACQUITY UPLC HSS T3 (100×2.1 mm×1.8 μm) with Acquity UPLC. The mobile phase was composed of solvent A (0.05% formic acid water) and solvent B (acetonitrile) with a gradient elution (0-1.0 min, 5% B; 1.0-12.5 min, 5%-95% B; 12.5-13.5 min, 95% B; 13.5-13.6 min, 95%-5% B; 13.6-16.0 min, 5% B). The flow rate of the mobile phase was 0.3 mL/min. The column temperature was maintained at 40° C., and the sample manager temperature was set at 4° C.

Mass spectrometry parameters in ESI+ and ESI-mode were following: ESI+: Heater Temp 300° C.; Sheath Gas Flow rate, 45arb; Aux Gas Flow Rate, 15arb; Sweep Gas Flow Rate, larb; spray voltage, 3.0 KV; Capillary Temp, 350° C.; S-Lens RF Level, 30%.

ESI−: Heater Temp 300° C., Sheath Gas Flow rate, 45arb; Aux Gas Flow Rate, 15 arb; Sweep Gas Flow Rate, larb; spray voltage, 3.2 KV; Capillary Temp, 350 CC; S-Lens RF evel, 60%.

Based on the analysis, the samples contained over 1004 proteins and over 6000 related peptides and over 500 metabolites. Surprisingly, neither VEGF, IGF-1 nor FGF-2 was among the identified proteins or peptides.

Instead, the samples contained various lipids, associated proteins and proteins related to lipid metabolism; various basement membrane components and proteins and peptides related to dermo-epidermal junction or proteins and peptides affecting dermis and epidermis; essential and non-essential amino acids, antioxidants, vitamins, and larger number of glycoproteins and proteoglycans as well as factors related to cell proliferation. Some of the compounds identified are regarded as endogenous, some exogenous. However, all the component originate from the donor tissue. It also to be noted that some of the components are intracellular, some extracellular.

Some of the identified molecules are listed in Table 1 below.

TABLE 1

| | |
|---|---|
| Structural and non- | Collagens I, IV, VI, XV, XVIII (e.g. Collagen alpha-1(I) chain, |
| structural | Collagen alpha-3(VI) chain, Collagen alpha-1(XVIII) chain, |
| extracellular matrix | Collagen alpha-1(XV) chain), Fibronectins, vitronectin, decorin, |
| proteins and proteins | laminins (e.g. Laminin subunits beta-1, Laminin subunit beta-2, |
| related to the synthesis | Laminin subunit gam Laminin subunit alpha-4), Connective tissue- |
| of ECM | activating peptide III, Biglycan, Tenascin-X, Fibulin-1, Lumican, |
| | Prolargin, EGF-containing fibulin-like extracellular matrix protein 1, |
| | Periostin, Transforming growth factor-beta-induced protein ig-h3, |
| | Asporin |
| Lipids or associated | Ceramides (e.g. Cer 18:0;2O/14:0, Cer 8:1;2O/26:3, CerP |
| proteins | 18:1;2O/2:0, CerP 19:0;2O/2:0), Apolipoproteins (e.g. |
| | Apolipoprotein L1, Apolipoprotein M, Apolipoprotein A-I, |
| | Apolipoprotein E, Apolipoprotein A-II, Apolipoprotein C-I, |
| | Apolipoprotein C-II, Apolipoprotein C-III, Apolipoprotein B- |
| | 100; Apolipoprotein B-48, Apolipoprotein D, Apolipoprotein A-IV, |
| | Apolipoprotein F), Adipogenesis regulatory factor, Fatty acid- |
| | binding protein, Fatty acid synthase, Galectin-3, Long-chain-fatty- |
| | acid--CoA ligase 1, Phospholipid transfer protein, Serine |
| | palmitoyltransferase 3, Serine palmitoyltransferase 2, Perilipin-1, |
| | Perilipin-3, Perilipin-4, clusterin |
| | Fatty acids (medium and long chains, saturated and unsaturated): |
| | (e.g. 5-phenylvaleric acid, Adipate, Maleamic acid, Palmitic Acid, |
| | Palmitoleic Acid, Tetradecanedioic acid, Pentadecanoic acid, |
| | Tridecanoic acid, Tridecylic acid, Undecanoic acid, Undecanedioic |
| | acid) |
| | Monoglycerophospholipids (e.g. LPC 10:0, LPC 10:0-SN1, LPC |
| | 11:0, LPC 11:0-SN1, LPC 12:0, LPC 12:0-SN1, LPC 13:0, LPC 14:0, |
| | LPC 14:0-SN1, LPC 16:0, LPC 16:1, LPC 16:1-SN1, LPC 16:4-SN2, |
| | LPC 18:0, LPC 18:1, LPC 18:2, LPC 20:4, LPC 20:4-SN1, LPC O- |
| | 16:1, LPC O-16:2, LPC O-18.1) |
| | Prostaglandins: Prostaglandin F2, Prostaglandin F2α 1-11-lactone |
| | Prostaglandin F3, Prostaglandin E synthase 3 |
| | Sebacic acid |
| | Lysophosphatidic acids (e.g. LPA 12:0, LPA 13:0) |
| | Lysophospholipids (e.g. LPE 14:0, LPE 18:0, LPE 18:1, LPE 18:2, |
| | LPE 20:4) |
| | Lipid metabolism related proteins (e.g. Glycerol-3-phosphate |
| | dehydrogenase 1-like protein, Glycerol-3-phosphate dehydrogenase) |
| | Glycerophospholipids (e.g. PC 12:0_12:0, PC 18:0_18:1, PC |
| | 18:1_18:1, PC 34:1; PC 16:0-18:1, PC O-12:0, PC O-36:2, |
| | PC(16:0/0:0), PA 22:3; PA 6:0-16:3) |
| | Diacylglycerols (e.g. DG 28:2, DGDG 30:5; DGDG 12:0-18:5, |
| | DGDG O-9:0_22:4) |
| Basement membrane/ | Keratin type II, ceramides, Versican, Periplakin, Collagen alpha- |
| Dermo-epidermal | 1(XV) chain, Integrin-linked protein kinase, periplakin, Nidogen-1, |
| junction components, | Nidogen-2, Integrin beta-8, Prolargin, Basement membrane-specific |
| factors effecting | heparan sulfate proteoglycan core protein, Plectin, SUMO-activating |
| skin/epidermis | enzyme subunit 2, Talin-1, Talin-2 |
| Amino acids | Non-essential amino acids (e.g. Asparagine, Cysteine, Isodityrosine, |
| | Methionine, Taurine, Tyrosine, Pipecolic acid |
| | Essential amino acids (e.g. Threonine, Isoleucine, Leucine, |
| | Tryptophan, Norleucine, Norvaline) |
| Vitamins and peptides | Vitamin A related: Tretinoin, Retinal dehydrogenase 1, Retinol- |
| with antioxidant or | binding protein 4, Transthyretin; Vitamin D related: Vitamin D- |
| oxidoreductase | binding protein; Vitamin B related: Nicotinamide (Vitamin B3), |
| properties or related | Biotinidase, Pyridoxamine, Pantothenic acid; |
| molecules/ | Selenoprotein P, Pyroglutamic acid |
| metabolites | |

### Example 2. Safety Study

The purpose of the safety study was to evaluate the tolerability of the present allogenic lipoaspirate-derived preparation and to reveal possible mid-term adverse events (AEs) related to its use.

Three independent batches disclosed in Example 1 were used in the study to ensure the uniformity of the preparations also in the safety perspective.

The study was conducted as a randomized, double-blinded, placebo-controlled study at a private sector hospital, Helsinki, Finland, with signed informed consent form the study subjects.

To this end, a total of 38 volunteers (74% women) were recruited. The subjects represented all five skin types according to the Fitzpatrick classification, with an emphasis on categories two and three. 34% of the study subjects had some underlying disease such as hypertension, migraine or asthma, the rest were considered totally healthy. No one had an underlying disease that could affect the interpretation of the results. Subjects who had previously been diagnosed with an allergic reaction leading to anaphylaxis, as well as those with acute cancer were excluded from the study. Moreover, the subjects were not allowed to use any medication to treat allergies or resistance during the study to guarantee the reliability of the results.

Subcutaneous injections were chosen as the route of administration as that administration route is generally associated with a higher risk of AEs as compared to topical administration. A high dose (3-10 times the amount intended for normal use) of the test preparation was injected into the subcutaneous tissue of each subject's arm. The other side of the arm was injected with a corresponding amount of Ringer-Acetat solution, which served as a placebo. Ringer Acetat has not been associated with AEs and is generally considered as safe and routinely used for intravenous administration.

The study subjects were monitored for three months using an electronic diary, calls by research nurse and doctor's visits. The follow-up was initially daily and then monthly. The study was carried out in cooperation with a clinical research unit of a private sector hospital and was monitored by a independent contract research organization (CRO).

Both allergic reactions and irritation symptoms were extensively investigated in the study. All adverse reactions were reported, evaluated by the physician, and analyzed. The subjects measured their body temperature every day for a week, and none reported fever that was related to the administration of the test preparation. Also, no injection site infections were observed during the study. Immediately after the injection, the test preparation was found to cause slightly more redness at the injection site than the placebo.

Based on some earlier studies, the test preparation was known to cause temporary vasoconstriction, i.e., pale skin color in the injection area due to constriction of blood vessels. This effect is related to the preparation's mode of action and is not considered an AE or does not predict later AEs. Vasoconstriction events were reported also during the safety study.

Swelling and bruising were observed more related to the placebo injections than to the test injection, indicating that these reactions were primarily related to the route of administration (injection) and fluid accumulation into the tissue. Reported acute AEs are summarized in Table 2 below,

TABLE 2
Acute adverse reactions

| | Number of reported | Test | |
|---|---|---|---|
| Day | reactions | preparation | Placebo |
| Day 0 (n = 38) | Hematoma | 7 | 11 |
| | Erythema | 3 | 1 |
| | Edema | 2 | 2 |
| | Tingling | 1 | 0 |
| | Abnormal injection mark | 1 | 0 |
| | Injection site pain | 4 | 2 |
| Day 1 (n = 37) | Hematoma | 12 | 19 |
| | Erythema | 1 | 0 |
| | Edema | 1 | 1 |
| | Injection site pain | 2 | 4 |
| Day 2 (n = 37) | Hematoma | 11 | 18 |
| | Erythema | 1 | 0 |
| | Edema | 0 | 1 |
| | Injection site pain | 0 | 1 |
| Day 3 (n = 38) | Hematoma | 10 | 20 |
| | Erythema | 1 | 1 |
| | Edema | 0 | 1 |
| Day 4 (n = 38) | Hematoma | 9 | 18 |
| | Erythema | 1 | 1 |
| | Edema | 0 | 1 |
| Day 5 (n = 38) | Hematoma | 8 | 18 |
| | Erythema | 0 | 1 |
| | Abnormal injection mark | 0 | 1 |
| Day 6 (n = 37) | Hematoma | 6 | 15 |
| | Erythema | 1 | 1 |
| Day 7 (n = 38) | Hematoma | 7 | 15 |
| | Erythema | 1 | 0 |

In the three-month follow-up, no surprising or serious AEs were observed and nothing abnormal was observed in the doctor's assessment at day 90. The product was found to cause no mid-term AEs at all, particularly evaluated in terms of pigmentation, induration, necrosis, granuloma, or other abnormalities. No significant differences were found between the three batches used in terms of AEs, confirming that the preparation of the invention has a uniform safety profile with no batch-to-batch variation.

### Example 3. Improved Overall Skin Healing and Regeneration after Laser

Cell-free lipoaspirate-derived preparations disclosed in Example 1 were tested for their ability to improve overall healing of the skin and improve scar appearance after laser resurfacing with eight volunteers (aged 22-72 years) who were treated essentially in the same manner as the first subject.

FIG. 1 shows photographs of right and left profiles of a male (age 22) whose temple and cheek areas were treated with fractional CO2-laser to reduce the appearance of acne scars. Both sides of the face were treated with the same settings. After the treatment, the present cell-free lipoaspirate-derived preparation (0.5 ml) was topically applied to the left side of the face (the photograph on the right), whereas the right side of the face was treated with sterile water (the photograph on the left). The photographs illustrate the healing and re-epithelization process 2 days after the treatment. Improved recovery and faster healing on the area treated with the preparation of the invention was observed.

FIG. 2 shows photographs of a four times operated leg. A three year old scar was treated with fractional CO2 laser using the same settings for the whole scar. The lower half of the scar was treated twice by topical administration of the present cell-free lipoaspirate-derived preparation (0.5 ml), first immediately after the laser treatment and then 6 hours later for the second time. At 6h time point, vasoconstriction was seen in the scar area treated with the preparation of the invention. 2 Days after the laser resurfacing, the scar area treated with the preparation of the invention was less red and rough as compared to the non-treated area. 28 Days after the laser resurfacing, the area scar area treated with the preparation of the invention was much was lighter in color, softer and more at the skin level as compared to the non-treated scar area.

The other six subjects also reported faster recovery and less pain in the treated areas.

### Example 4. Promotion of Skin Regeneration in Microneedled Skin

Cell-free lipoaspirate-derived preparations disclosed in Example 1 were tested on three healthy volunteers with signed informed consent, who underwent standard facial microneedling, a widely used cosmetic procedure that is used to encourage collagen and elastin production using small, sterilized needles, for improving the appearance and texture of the skin. The follow up time was 4 weeks.

The first subject was a 34 year old female with 13 years old facial scars that had been treated earlier with microneedling without any improvement. Now the whole face, including the scar area was treated with microneedling (Dermapen 0.7 mm −2 mm depth) and right after that the preparation of the invention was applied topically onto the left side of the face (ca. 1 ml). VISIA pictures were taken prior to the treatment using standard angle and light.

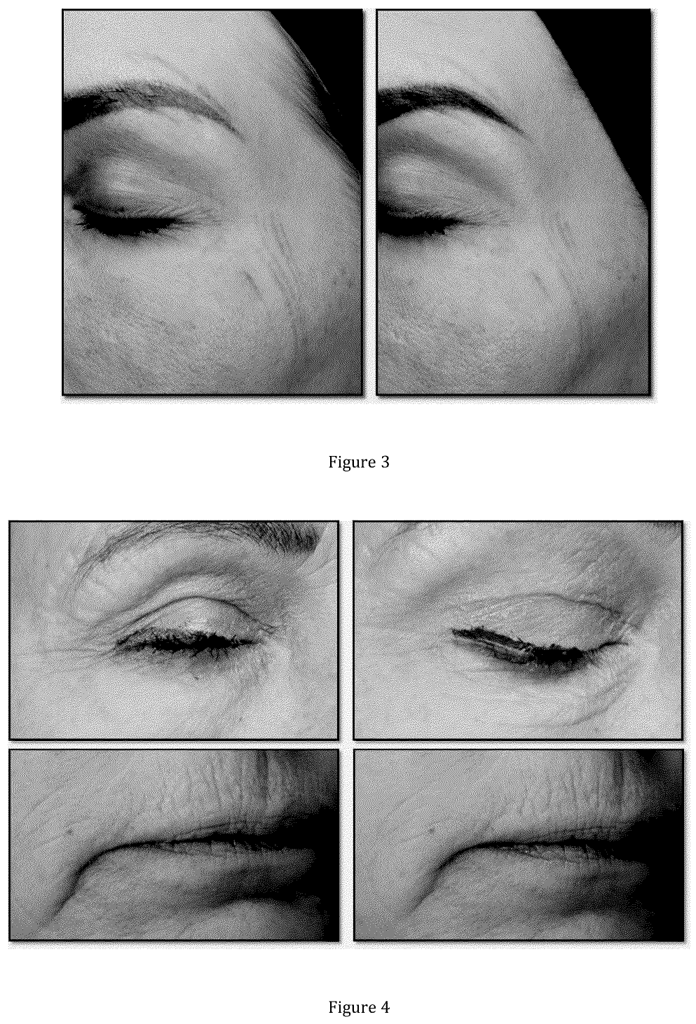

At a control visit 4 weeks after the treatment, the first subject reported significant improvement in the appearance of the scar area. Before and after photographs are shown in FIG. 3. At the same visit, new VISIA pictures were taken again using standard angle and light, and compared to the previous pictures. According to the VISIA analysis, patient showed improvement in skin texture, pores and red areas up to 31%.

The other two subjects (aged 59 and 72) were treated essentially in the same manner as the first subject. In VISIA analysis, they showed improvement in texture, pores and red areas up to 25%.

All subjects reported excellent outcomes in subjective evaluation.

### Example 5. Mesotherapy Treatments of Aged Skin

Cell-free lipoaspirate-derived preparations prepared as described in Example 1 were tested for their skin regeneration and/or rejuvenation potential on three healthy volunteers with signed informed consent. The subjects had non-compromised but aged skin. Each subject was treated with a different batch of the three batches prepared.

The first subject was a 58-old female who was treated with mesotherapy (sometimes called intradermatherapy or multi-puncture treatment). Circa 70 papulas (volume 0.04 ml/papula) were injected to the whole face area. Four weeks after the treatment, significant improvement in skin quality (soft, “velvet-kind of” texture) was observed subjectively as well as in a VISIA picture (standards angle and light). In addition, vanishing of vertical lines around the mouth area as well as in the eye area was observed. According to the VISIA analysis, texture and spots were improved up to 23%. In overall VISIA analysis, skin age was decreased for two years. Before and after photographs are shown in FIG. 4.

The two other subjects (aged 67 and 70) were treated essentially in the same manner as the first subject. In VISIA analysis, texture and UV spots were improved up to 7% and skin age decreased up to two years.

All subjects reported excellent outcomes in subjective evaluation.

The second treatment was performed 6 weeks after the first one. Three weeks after the second treatment, the subjects were still very pleased and reported excellent outcomes in subjective evaluation.

### Example 6. Intradermal Injection Treatment for Aging Skin

Cell-free lipoaspirate-derived preparations prepared as described in Example 1 were tested for their skin regeneration and/or rejuvenation potential on fifteen subjects with median age of 53 years using an intradermal injection treatment essentially as described in Example 5. The subjects had non-compromised but aged skin. Areas treated were face, neck and decollete. The subjects made subjective evaluation of their skin 2 months after treatment. They evaluated their sensation on the skin and skin appearance on a scale 1-6. (1=weaker than before treatment, 2=no change, 3=better than before treatment, 4=much better than before treatment, 5=met expectations, 6=great result/expectations were exceeded). The results are shown in Table 3. The subjects were overall experiencing that the treatment gave more than satisfactory results, sensation and appearance on the skin being much better than before the treatment. Especially patients with decollete treatment reported high satisfaction. At four months, when 7 of the patients had been re-evaluated, subjects reported equal satisfaction on the sensation on the skin and increased satisfaction towards the skin appearance as compared to the corresponding evaluations after two months from the treatment.

TABLE 3

| Parameter | | Mean | Median |
|---|---|---|---|
| Age | | 52.8 | 53 |
| Sensation | Smoother skin texture | 4.1 | 4 |
| on the skin | Softer skin | 4.1 | 3.5 |
| | Firmer skin structure | 4.1 | 4 |
| | Increased elasticity | 4.2 | 4 |
| | Generally better skin function and condition | 4.5 | 5 |
| Skin | Increased brightness and more even skin tone | 3.9 | 4 |
| appearance | Reduction of visible lines and wrinkles | 4.2 | 4.5 |
| | Increased fullness (volume) | 4 | 4 |
| | Generally more fresh and cheerful skin/look | 4 | 4 |

### Example 7. Injection Treatment for Acne Scars

Several subjects with old acne scars were treated with injections into dermis with cell-free lipoaspirates, prepared essentially as described in Example 1, to the area of the acne scars reaching the full length and depth of the acne scarred areas of the face, with techniques commonly used with injection treatments of acne scars. The results were surprisingly good already after 3-7 weeks from the onset of the treatments. The subjects observed that the treatment with the cell-free lipoaspirate started to affect almost immediately. Dark pigmentations started to disappear and starting from 3 weeks onwards, the skin felt more healthy, acne scars had become less visible and less deep and softer. At 7 weeks, the skin felt healthier, more elastic and scars no longer felt like hard areas on the skin. Scars had softened considerably and they felt less tight with facial movements. The surface of the skin was smoother, the scar pits and the blistering areas had subsided.

### Example 8. Fibroblast Migration and Induced ECM Protein Expression

BJ fibroblasts (ATCC, CRL-2522), were used to study migration and altered gene expression following exposure to a cell-free lipoaspirate-derived preparation prepared essentially as described in Example 1. In both cases BJ fibroblasts were cultured in GlutaMAX™ supplemented MEM (Gibco, REF: 41090036) with 10% FBS (Gibco, A3160501), 1x MEM Non-Essential Amino Acids Solution (Gibco, REF: 11140050) and 1% Antiobiotic-antimycotic (Gibco, 15240096). For migration, 20 000 cells were plated on each well on 3 well cell culture-inserts (Ibidi, REF: 80369) to reach confluent cell density, while for qPCR cells were plated in density of 20 000 cells/cm² on 6 well Nunc™ cell-culture treated multidishes (Thermo Scientific, REF 140685). In both experiments the cells were plated on day −1 and the cell-free lipoaspirate-derived preparation was added on day 0. In the exposures half of the total plating volume was the complete cell culture media. In the vehicle controls, the other half was Ringer's solution while in the substance exposures the protein concentration of cell-free lipoaspirate-derived preparation was calculated and based on that 1 mg/ml of cell-free lipoaspirate-derived preparation was added with Ringer's solution to fill the full plating volume. For migration two replicate inserts were used for vehicle control and substance exposures, resulting in 4 replicates in total with 3 well inserts, while for qPCR 3 replicates were used.

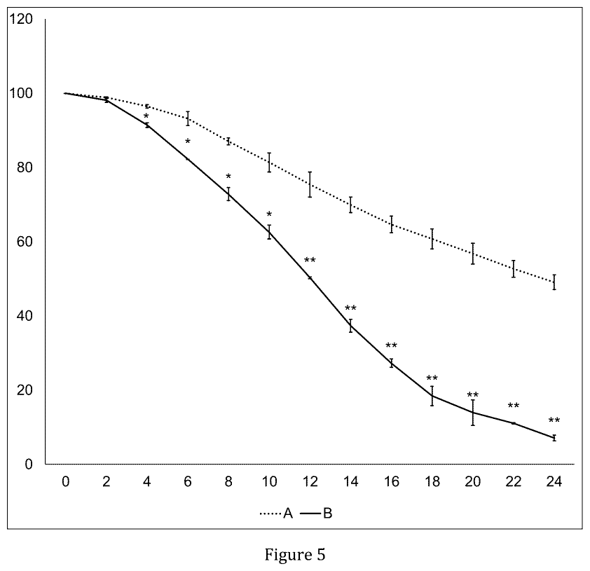

The migration was imaged in time-laps imaging with Leica DMi8 microscope, which was set to take images every hour for 24 h. The remaining area between cell culture-insert wells was measured in ImageJ 1.54f using Labkit plugin to define cell free area. For the clarity of the results, only every two hours are shown in FIG. 5. The results show a significant increase in fibroblast migration following cell-free lipoaspirate-derived preparation exposure starting from hour 4 and lasting until the end of the experiment duration. Increased fibroblast migration is commonly known to play a role in wound healing and skin regeneration processes, thus these results support cell-free lipoaspirate-derived preparations ability to induce skin regeneration and wound healing by attracting fibroblasts to the site of tissue damage.

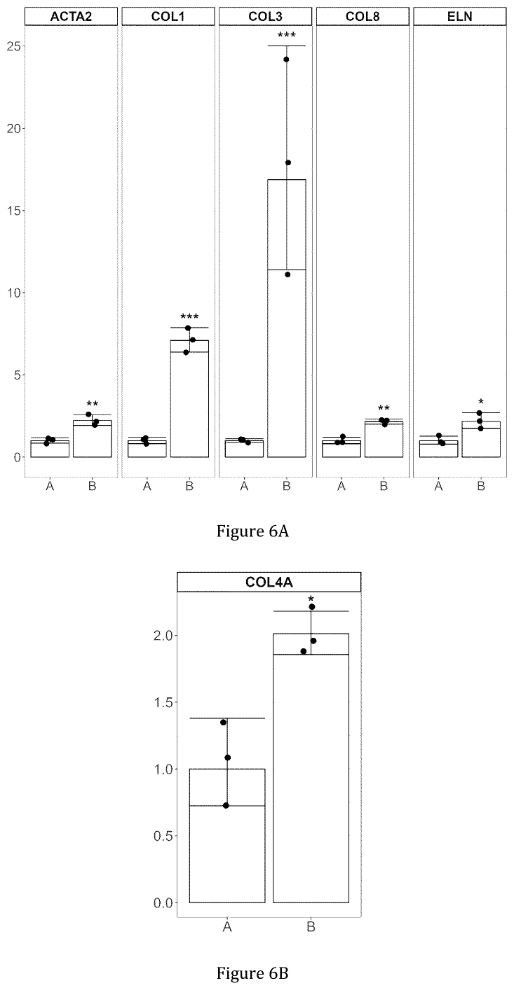

RNA for qPCR was purified on day 6 of exposure with GeneJET RNA Purification Kit (Thermo Scientific, K0732). Same concentration of RNA from all samples was translated to cDNA using Maxima™ H Minus cDNA Synthesis Master Mix (Thermo Scientific, M1681) with dsDNAse treatment. Gene expression was analyzed with TaqMan Real-Time PCR assays for smooth muscle actin (ACTA2), collagen 1 (COL1), collagen 3 (COL3), collagen 8 (COL8), elastin (ELN) and in following experiment for collagen 4 A (COL4A) with importin 8 (IP08) used as a housekeeping gene in both runs with TaqMan™ Universal PCR Master Mix (Applied Biosystems, 4304437). PCR was performed using CFX96 Real-Time PCR system (Bio-Rad). The forementioned collagens and elastin are important components of dermal extracellular matrix (ECM) and basal layer, supporting healthy skin structure, while ACTA2 is indicative of fibroblast differentiation into myofibroblasts which produce ECM components. The qPCR results (FIG. 6 A and FIG. 6 B) display induced expression of all measured components supporting cell-free lipoaspirate-derived preparations ability to remodel ECM.

### Example 9. Human Adipose Stem Cell Differentiation

Primary human adipose stem cells (hASC) were used to model mesenchymal stem cells of the skin and their differentiation. The differentiation was measured with AdipoRed™ (Lonza, PT-7009) triglyceride accumulation assay and qPCR gene expression analysis. The cells were cultured in DMEM/F12 supplemented with GlutaMax (Gibco, A41920-01), 10% human serum (BioSell) and 1% Antiobiotic-antimycotic (Gibco, 15240096). For both AdipoRed and qPCR, 22 000 cells/cm² were plated, for AdipoRed in 48 well plate and for qPCR on 6 well plate, both in three replicates. For qPCR the exposures were done similarly to Example 8 with 1 mg/ml while with AdipoRed the exposures were 0.8 mg/ml and 0.4 mg/ml with half of the total volume consisting complete cell culture media filled to total volume with Ringer's solution.

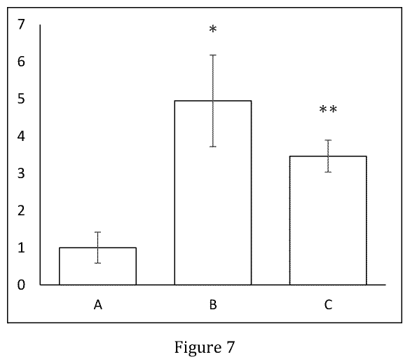

AdipoRed measurement was done following manufacturer's instructions. Briefly, AdipoRed was diluted into 3% mixture with PBS (Gibco, REF). Cells were washed with RT PBS and after which they were incubated in 3% AdipoRed for 15 min. Fluorescence was measured with Tecan Spark multimode plate reader with excitation wavelength of 485 nm and emission wavelength of 572 nm. Cell-free lipoaspirate-derived preparation induced dose-dependent increase in triglyceride production, where both concentrations induced significant increase of signal (p. value <0.05) (FIG. 7). The accumulation of triglyceride is indicative of differentiation of mesenchymal stem cells.

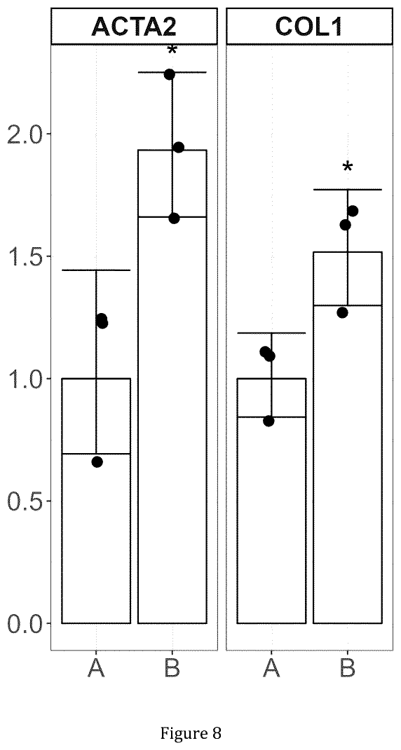

RNA purification, cDNA translation and qPCR were done similarly to Example 8 after 6 days of cell-free lipoaspirate-derived preparation exposure. The TaqMan assays used were ACTA2 and COL1 with IP08 as housekeeping gene. The results show induced expression of both ACTA2 and COL1 in the samples exposed to cell-free lipoaspirate-derived preparations in comparison to vehicle control (FIG. 8). These changes are indicative of hASC differentiation into specialized cell type such as skin cell types (e.g. endothelial cells and fibroblast-like cells).

The results support the skin regeneration and anti-fibrotic potential of the present cell-free preparation.

### Example 10. Keratinocyte Proliferation and Migration

NHEK-neo (Lonza, 00192906), pooled neonatal human epidermal keratinocytes were used to study keratinocyte proliferation and migration. The cells were grown in KBM™ Gold™ Basal Medium (Lonza, 00192151) supplemented with KGM™ Gold™ SingleQuots™ (Lonza, 00192152). For the proliferation assay, the cells were cultured in density of 10 000 cells/cm² on 48-Nunc™ cell-culture treated multidishes (Thermo Scientific, 150687). In the migration assay, 40 000 cells were plated on each well on 3 well cell culture-inserts (Ibidi, REF: 80369) in two replicates. The exposures in both proliferation and migration assays were done similarly to Example 8, where both vehicle control and cell-free lipoaspirate-derived preparation exposures had complete cell culture medium filling half of total exposure volume and the exposures had 1 mg/ml of cell-free lipoaspirate-derived preparation filled to total volume with Ringer's solution.

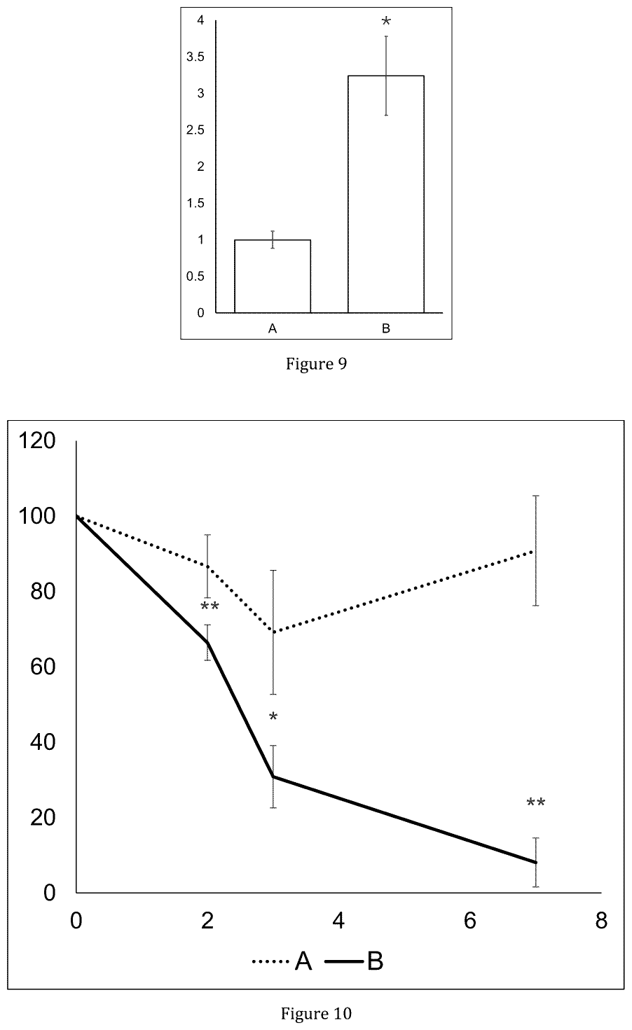

Proliferation was measured on day 6 by counting nucleai stained with Hoechst 33342 (Invitrogen, H3570). Cells were washed with PBS and incubated for 10 minutes in Hoechst diluted 1:1000 in PBS. Cells were imaged with excitation wavelength of 350 nm and emission wavelength of 461 nm. The nuclei were calculated in ImageJ 1.54f. The results (FIG. 9) show a significant increase in the proliferation of the cell-free lipoaspirate-derived preparation treated cells.

Migration progression was imaged on days 0, 2, 3 and 7 with Leica DMi8 microscope. The cell free area of images was analyzed using ImageJ 1.54f using Labkit plugin. Migration was significantly increased (p. value <0.05) with cell-free lipoaspirate-derived preparation treated samples on days 2, 3 and 7, where on day 7 the two cell populations from different insert sectors were fully merged (FIG. 10).

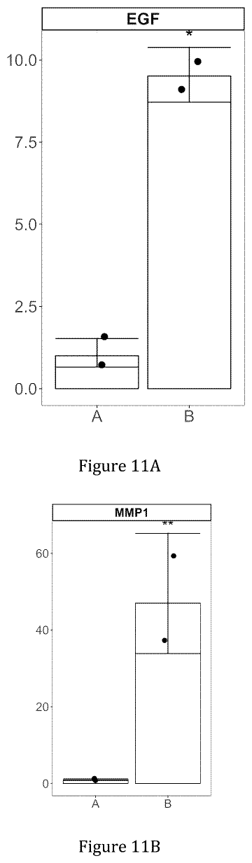

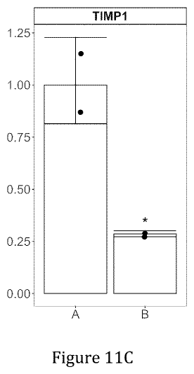

The cells were left on the migration plate for 8 more days, after which RNA was extracted. During the growing of cells cell culture media and exposures were changed three times, on days 3, 7 and 10. At this point the cell populations in vehicle controls was still separated and still fully merged in cell-free lipoaspirate-derived preparation treated samples. From these samples RNA was purified using GeneJET RNA Purification Kit (Thermo Scientific, K0732). RNA was dsDNAse treated and translated to cDNA using similar methods as in example 8. qPCR was run with TaqMan assays for epidermal growth factor (EGF), matrix metalloproteinase (MMP1), and TIMP Metallopeptidase Inhibitor 1 (TIMP1). Matrix metalloproteinase is needed for cell migration for the cell to move through extracellular matrix, which can be inhibited by TIMP. EGF on the other hand induces keratinocyte proliferation and migration. The qPCR analysis showed increased gene expression of EGF and MMP1 and decreased expression of TIMP1 (FIGS. 11A, 11B and 11C). Altogether the increased proliferation, migration and EGF and MMP1 gene expression with decreased expression of TIMP1 indicate increased keratinocyte turn-over induced by the cell-free lipoaspirate-derived preparation. Additionally, EGF has various indications related to skin regeneration and healing of skin diseases such as acne, atopic dermatitis, wounding and scarring.

### Example 11. Enrichment Analysis of Proteomics

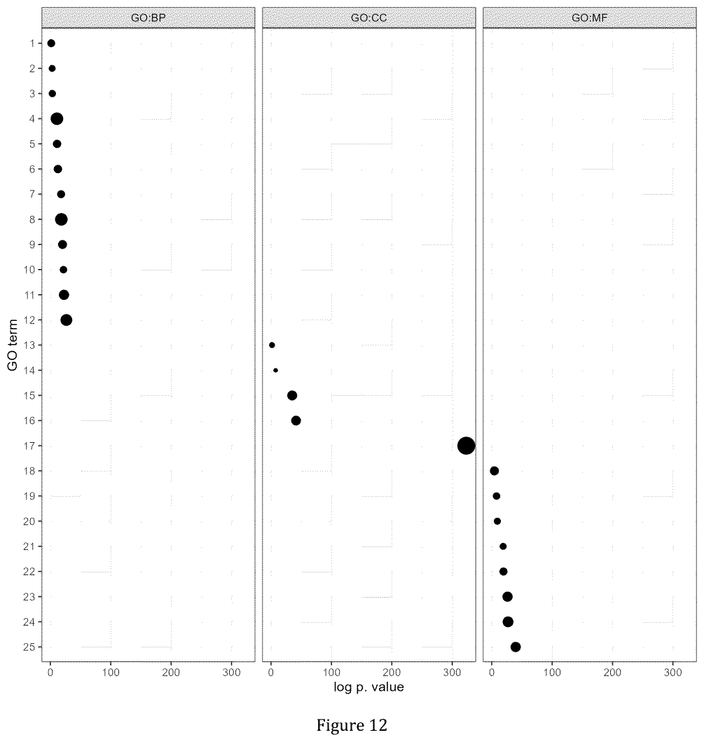

New LC-MS proteomics analysis was performed with similar method as in Example 1 but on this time with cell-free lipoaspirate-derived preparations derived from 10 different donors in three replicates. The gene-coding proteins, identified in untargeted proteomics analysis, were further investigated by functional enrichment using g:Profiler enrichment tool calculating enrichments for Gene Ontology (GO) knowledge base. The enrichement analysis identified 1025 significantly enriched terms across GO sub-ontologies; 178 molecular functions (GO:MF), 691 biological processes (GO:BP) and 156 cellular compartments (GO:CC). Of these terms sub-selection was made by going through the terms manually and selecting descriptive terms by ignoring highly specific or pervasive child and ancestor terms. This selection resulted in 25 terms displayed in FIG. 12.

Overall 12 terms were selected from GO:BP (Biological processes). These terms include angiogenesis, tissue migration, wound healing, immune system related processes, proteolysis activity cellular component organization and cellular detoxification, which all are relevant processes to tissue healing and regeneration. In GO:CC (cellular compartment) 5 terms were selected including immunoglobulin complex, high density lipoprotein particle, ECM related terms and extracellular exosomes. These terms give a general overview of building blocks in the cell-free lipoaspirate-derived preparations. Finally, 8 GO:MF (Molecular function) terms were selected highlighting antioxidant, oxidoreductase and peptidase regulatory activities of the proteins in the cell-free lipoaspirate-derived preparation as well as terms related to protein-binding affinities to glycosaminoglycans, cytoskeleton and cell adhesion molecules.

1. A cell-free lipoaspirate-derived preparation, wherein the preparation comprises at least one of a lipid fraction and an aqueous fraction of a lipoaspirate.

2. The cell-free lipoaspirate-derived preparation according to claim 1, wherein the aqueous fraction comprises spent liposuction solution, with or without a local anesthetic and/or adrenaline.

3. The cell-free lipoaspirate-derived preparation according to claim 1, wherein the preparation comprises the aqueous fraction of the lipoaspirate and the lipid fraction of the lipoaspirate as the only liposuction-derived material in the preparation.

4. The cell-free lipoaspirate-derived preparation according to claim 1, wherein the preparation comprises the aqueous fraction of the lipoaspirate, the lipid fraction of the lipoaspirate, and a spent liposuction solution not collected as part of the lipoaspirate as the only liposuction-derived material in the preparation.

5. The cell-free lipoaspirate-derived preparation according to claim 1, which has been filtered through a membrane filter, having a pore size of about 0.2 μm or about 0.22 μm.

6. The cell-free lipoaspirate-derived preparation according to claim 1, wherein the preparation comprises at least one component selected from the group consisting of structural and non-structural extracellular matrix proteins, peptides, chains, subchains and subunits thereof; proteins related to the synthesis of extracellular matrix; lipids, lipid-associated proteins, proteins related to lipid metabolism; basement membrane proteins peptides, chains, subchains and subunits thereof; specialized dermo-epidermal junction components and factors effecting epidermis; essential and non-essential amino acids; antioxidants; vitamin derivatives and metabolites; or glycoproteins and proteoglycans.

7. The cell-free lipoaspirate-derived preparation according to claim 6, wherein:
i) the structural and non-structural extracellular matrix proteins, peptides, chains, subchains and subunits thereof include at least one component selected from the group consisting of collagens I, III, IV, VI, XV, XVIII, fibronectin, vitronectin, elastin, hyaluronan, decorin, tenascin, laminin, lumican and prolargin; and/or
ii) the lipids, lipid-associated proteins, proteins related to lipid metabolism include one or more components selected from the group consisting of ceramids, apolipoproteins, perilipins, non-saturated and saturated fatty acids, glycerophospholipids, lysophosphatidic acids, lysophospholipids, monoglycerides, diglycerides, triglycerides and prostaglandins; and/or
iii) the basement membrane and dermo-epidermal junction proteins, peptides, chains, subchains and subunits thereof include one or more component selected from the group consisting of keratin, nidogen, versican, intergrins, periplakin, plectin; and/or
iv) the vitamin derivates and metabolites include at least Tretinoin.

8. The method of producing the cell-free lipoaspirate-derived preparation according to claim 1, comprising the steps of:
providing a human lipoaspirate;
removing an adipose tissue fraction of the lipoaspirate, said fraction comprising cells; and
filtering the remaining fractions of the lipoaspirate.

9. The method according to claim 8, wherein prior to removing the adipose tissue fraction, the lipoaspirate is allowed separate into a lipid first layer, an aqueous second layer, and an adipose tissue layer comprising adipocytes and other cells of the adipose tissue interposed between the first layer and the second layer.

10. The method of producing the cell-free lipoaspirate-derived preparation according to claim 1, comprising the steps of:
providing a human lipoaspirate;
collecting at least one of a lipid fraction and an aqueous fraction, while discarding an adipose tissue fraction comprising cells; and
filtering the collected fraction(s).

11. The method according to claim 10, wherein prior to collecting at least one of the lipid fraction and the aqueous fraction, the lipoaspirate is allowed to settle such that the lipoaspirate separates into a lipid first layer, an aqueous second layer, and an adipose tissue layer comprising adipocytes and other cells of the adipose tissue interposed between the first layer and the second layer.

12. The method according to claim 9, wherein the method further comprises omitting rinsing of the lipoaspirate or any fraction thereof.

13. The method according to claim 9, wherein the filtering is carried out by using a membrane filter having a pore size of about 0.2 μm or about 0.22 μm.

14. The cosmetic or pharmaceutical composition comprising the cell-free preparation according to claim 1 and a physiologically acceptable carrier, adjuvant and/or excipient.

15. The cosmetic or pharmaceutical composition according to claim 14, wherein the composition is in the form of an ointment, lotion, cream, gel, hydrogel, oil-in-water emulsion, water-in-oil emulsion, drop, spray, liquid, solution, powder, slow release or sustained release formulation, face mask, skin patch, mousse or foam.

16. The cosmetic or pharmaceutical composition according to claim 14, wherein the composition is administrable topically, subcutaneously, intradermally or intrahypodermally.

17. The method for treating a skin area of a human subject, the method comprising:
administering a cell-free lipoaspirate-derived preparation comprising at least one of a lipid fraction and an aqueous fraction of a lipoaspirate, or a cosmetic or pharmaceutical composition comprising the cell-free preparation according to claim 1 and a physiologically acceptable carrier, adjuvant and/or excipient to the skin area topically, at least one of subcutaneously, intradermally or intrahypodermally.

18. The method according to claim 17, wherein at least one of:
the skin area comprises at least one of compromised skin pre-treated by microneedling, exfoliation and/or laser resurfacing; or
the method comprises subjecting the skin area to microneedling, laser resurfacing or exfoliation prior to the administering step.

19. The method according to claim 17, wherein the skin area of the human subject comprises at least one of aged skin, photodamaged skin, or scarred skin, and/or the skin area comprises at least one clinical skin condition selected from the group consisting of acne, actinic keratosis, atopic dermatitis, venous stasis dermatitis, eczema, basal cell carcinoma, contact dermatitis, keloids, lichen planus, melasma, vitiligo, psoriasis, rosacea, seborrheic dermatitis, chronic wounds such as diabetic wounds, burns, erythema multiforme, epidermolysis bullosa, lupus and keratosis pilaris.
